(19) United States
(12) Patent Application Publication
(10) Pub. No.: US 2022/0362546 A1
(43) Pub. Date: Nov. 17, 2022

(54) IMPLANTABLE MEDICAL DEVICE COMPRISING AN ANCHORING DEVICE

(71) Applicant: BIOTRONIK SE & Co. KG, Berlin (DE)

(72) Inventors: Thomas Doerr, Berlin (DE); Gernot Kolberg, Berlin (DE)

(73) Assignee: BIOTRONIK SE & Co. KG, Berlin (DE)

(21) Appl. No.: 17/772,303

(22) PCT Filed: Oct. 23, 2020
(86) PCT No.: PCT/EP2020/079845

§ 371 Date: Apr. 27, 2022

(30) Foreign Application Priority Data
Nov. 1, 2019 (EP) 19206696.7
Nov. 1, 2019 (EP) 19206704.9
Nov. 1, 2019 (EP) 19206718.9

(51) Int. Cl. A61N 1/05; A61N 1/375

(52) U.S. Cl. CPC A61N 1/0573; A61N 1/3756

(57) ABSTRACT

An implantable medical device for implantation into a patient comprises a body, an anchoring device for anchoring the body to tissue at a location of interest, the anchoring device being arranged on the body, and an electrode device for at least one of emitting an electrical stimulation signal and sensing an electrical sense signal. The electrode device comprises at least one electrode and a helically extending coil body, wherein the electrode device is movable with respect to the body between a retracted position, in which the electrode device at least partially is received within the body, and an engagement position, in which the electrode device is moved to protrude from the body to engage with tissue.

### CROSS-REFERENCE TO RELATED APPLICATIONS

This application is the United States National Phase under 35 U.S.C. § 371 of PCT International Patent Application No. PCT/EP2020/079845, filed on Oct. 23, 2020, which claims the benefit of European Patent Application No. 19206696.7, filed on Nov. 1, 2019, European Patent Application No. 19206704.9, filed on Nov. 1, 2019 and European Patent Application No. 19206718.9, filed on Nov. 1, 2019, the disclosures of which are hereby incorporated by reference herein in their entireties.

### TECHNICAL FIELD

The present invention relates to an implantable medical device for implantation into a patient according to the preamble of claim 1.

### BACKGROUND

An implantable medical device of this kind comprises a body, an anchoring device for anchoring the body to tissue at a location of interest, the anchoring device being arranged on the body, and an electrode device for at least one of emitting an electrical stimulation signal and sensing an electrical sense signal.

The implantable medical device may, for example, have the shape of a leadless stimulation device, such as a leadless pacemaker device. In this case the body is formed by the housing of the leadless pacemaker device, which encapsulates components of the leadless pacemaker device such as a processor, a data memory, a battery and other processing equipment to allow for operation of the leadless pacemaker device in an autarkic manner. The leadless pacemaker device may be implanted directly into the heart and may operate within the heart, for example within the right ventricle of the heart, without requiring any leads for placing an electrode at a location of interest within the heart.

Alternatively, the implantable medical device may be a stimulation device which comprises a generator to be implanted, for example, subcutaneously at a location remote from the heart. In this case the body is formed, for example, by a lead extending from the generator into the heart to allow for a stimulation or a sensing of signals at a location of interest within the heart, for example within the right ventricle.

With common electrode arrangements of implantable medical devices, an injection of stimulation signals generally is possible at the surface of intra-cardiac tissue, an electrode being in contact with intra-cardiac tissue in order to allow an injection of stimulation energy into the tissue. With new approaches, for example, for providing a stimulation in case of a so-called left bundle block, it may be desired to provide for an excitation in a localized fashion in the region of the so-called left bundle branch, which requires to engage with intra-cardiac tissue in the range of the septum of the heart and to place an electrode in the vicinity of the left bundle branch, such that stimulation energy may be specifically injected into the left bundle branch. As this requires a piercing of the septum, there is a general desire to provide for an anchoring of an implantable medical device on intra-cardiac tissue which is easy to establish and allows for a spatially differentiated excitation of tissue, in particular in the context of a left bundle branch pacing.

In particular, when introducing an electrode, for example, arranged on a lead from the right ventricle into the septum in order to reach towards the left bundle branch, the electrode must be inserted into the tissue to reach a substantial depth in order to come to lie in the vicinity of the left bundle branch. This comes with the inherent risk that a piercing structure on which the electrode is arranged may pierce through the septum and may reach into the left ventricle. In addition, the piercing itself possibly may have a significant impact on tissue and may even destroy tissue. In addition, when placing an electrode on the septum to reach to the left bundle branch, there is a risk of dislocation, for example when the electrode is arranged on an anchoring device in the shape of a screw, such that an electrical coupling in between the electrode and the left bundle branch may deteriorate over time due to, for example, a displacement of the screw, potentially leading to a capture loss.

International Publication No. WO 2008/058265 A2 discloses a cardiac stimulation system and method which allow to deliver a left ventricle stimulator from a right ventricle lead system in the right ventricle chamber, into a right side of the septum at a first location, and transmuscularly from the first location to a second location along the left side of the septum. The left ventricle stimulator is fixed at the second location for transmuscular stimulation of the left ventricular conduction system. A biventricular stimulation system further includes a right ventricle stimulator also delivered by the right ventricle lead system to the first location along the right side of the septum for right ventricular stimulation.

U.S. Publication No. 2009/0276000 discloses a method for delivering physiological pacing by selecting an electrode implant site for sensing cardiac signals, which is in proximity to the hearts intrinsic conduction system. An arrangement of multiple electrodes herein is arranged on a tip of a lead.

U.S. Pat. No. 10,406,370 discloses a device for providing cardiac pacing by multiple electrodes inserted using a single conduit. Acceptable electrodes herein are selected as active based on a predetermined criteria, and cardiac stimulation is provided for multiple chambers of the heart from a single location.

The present disclosure is directed toward overcoming one or more of the above-mentioned problems, though not necessarily limited to embodiments that do.

### SUMMARY

It is an object of the instant invention to provide an implantable medical device which allows for an easy implantation and operation, in particular in order to provide for a left bundle branch pacing operation.

At least this object is achieved for means of an implantable medical device comprising the features of claim 1.

Accordingly, the electrode device comprises at least one electrode and a helically extending coil body, wherein the electrode device is movable with respect to the body between a retracted position, in which the electrode device at least partially is received within the body, and an engagement position, in which the electrode device is moved to protrude from the body to engage with tissue.

The body may extend longitudinally along a longitudinal axis. The body herein may, for example, form a distal end to be placed on tissue upon implantation of the implantable medical device, the anchoring device being arranged on and extending from the distal end. In particular, the anchoring device may extend from the body generally along the longitudinal axis, the anchoring device protruding from the body in order to allow a piercing of tissue by means of the anchoring device in order to provide for an anchoring of the implantable medical device on tissue. In addition, the electrode device is movable with respect to the distal end, such that the electrode device may be adjusted in its position with respect to the distal end.

Generally, the electrode device in its retracted position is at least partially received within the body. Hence, in the retracted position the electrode device fully or at least in part is located within the body and hence is covered towards the outside by the body, such that for example, the implantable medical device may be implanted and for this may be moved towards a location of interest without the electrode device hindering an advancement of the implantable medical device towards the location of interest. Once the location of interest is reached, the electrode device can be moved out of the body such that the electrode device protrudes from the body and in particular can be brought into engagement with tissue at the location of interest, such that one or multiple electrodes arranged on the electrode device come into electrical contact with tissue and hence may couple with tissue in order to at least one of emitting an electrical stimulation signal into and sensing an electrical sense signal from tissue at the location of interest.

Because during implantation of the implantable medical device the electrode device may be moved with respect to the body and hence may be brought into an engagement position with respect to the body in which a particular target area, for example on the septum of the heart, may be targeted, at least one of the electrodes of the electrode device, for example, may be brought into a position in which the electrode device comes to rest in the region of, e.g., the left bundle branch and may electrically contact the left bundle branch. In this way a pacing at the left bundle branch may be provided, allowing for a physiological stimulation by achieving a propagation of stimulation signals along the bundle branches of the ventricles at low stimulation thresholds. A left bundle branch stimulation may allow for an easy and reliable implantation and easy stimulation algorithms, in particular not requiring a particular adjustment of AV delays as necessary, for example, for an HIS bundle pacing.

Implantation of the implantable medical device in particular becomes easy because within an implantation procedure the electrode device may be progressively advanced from the body to approach a depth in which the left bundle branch is located, wherein during implantation a capturing of the left bundle branch may be continuously monitored such that advancement of the electrode device may be stopped once an electrode of the electrode device couples with the left bundle branch. Hence, implantation may be easy, and operation may be reliable.

In addition, even when a dislocation of the electrode device occurs, this may not have an impact on the operation of the implantable medical device, as the electrode device may be relocated to again capture the left bundle branch to avoid a capture loss, such that operation may be modified and adapted over the operative lifespan of the implantable medical device.

In one embodiment, the electrode device is movable with respect to the body by a rotation, such that the electrode device with its helically extending coil body may be screwed into tissue at a location of interest in order to bring the electrode arranged on the electrode device into electrical contact with the tissue and to, for example, electrically couple to the left bundle branch at a substantial depth within tissue. The screwing action herein may be carried out when the body is mechanically coupled to tissue by means of the anchoring device, such that during the screwing operation the body is held in place and in particular is rotationally fixed with respect to the body, allowing to rotate the electrode device to screw the electrode device into tissue.

The electrode device herein may be moved out of the body to, e.g., assume arbitrary positions with respect to the body. This allows to bring the electrode device in such a position that an electrical coupling to a specific cardiac tissue region, for example the left bundle branch, may be obtained.

The electrode device herein may be continuously movable and may be stopped at arbitrary positions. Alternatively, different, discrete detent positions may be defined into which the electrode device can be moved, such that the electrode device can be brought into one of a multiplicity of different, discrete positions with respect to the body.

The electrode device may comprise multiple electrodes. Hence, a coupling to tissue may be achieved by one or multiple of the multiplicity of electrodes formed on the electrode device. If multiple electrodes are present on the electrode device, the electrodes may be electrically independent from each other such that a stimulation and/or sensing may be performed independently by one or multiple electrodes. By using multiple electrodes on the electrode device, one or a subset of the electrodes may be selected in order to couple to a specific tissue area, in addition to adjusting the position of the electrode device by mechanically moving the electrode device.

In one embodiment, the body may be formed by a lead which is connectable to a generator of the implantable medical device. In this case, the generator may be implanted into a patient, for example, subcutaneously remote from the heart, the lead forming the body extending from the generator into the heart such that the body with the anchoring device and the electrode device arranged thereon is placed in the heart, for example within the right ventricle in order to engage with tissue at the right ventricle. If the anchoring device and the electrode device are placed at the distal end of the body, the distal end is to be implanted into the heart to engage with intra-cardiac tissue for anchoring the body with its distal end on tissue within the heart. By engaging with tissue, herein, the electrode device couples with tissue and hence may be used to at least one of emitting an electrical stimulation signal and sensing an electrical sense signal.

The lead may, for example, comprise a connector which allows an electrical connection of the lead to the generator. The connector may, for example, be plugged into a corresponding plug of the generator, wherein the connector may, for example, comprise an arrangement of contact elements to electrically contact to the generator. The connector may have a standardized shape, and may, for example, be formed as a DF2 or DF4 connector.

In one embodiment, the connector comprises a gauge device for indicating the position of the electrode device with respect to the body. The gauge device may comprise a marker element which is linearly movable when moving the electrode device with respect to the body, such that according to the position of the marker element the relative position of the electrode device with respect to the body can be assessed. In an alternative embodiment, the gauge device may comprise a turning wheel by which the electrode device may be rotated with respect to the body, wherein the rotational position of the turning wheel indicates the relative position of the electrode device.

In another embodiment, the body may be formed by a housing of a leadless pacemaker device. In this case, the implantable medical device is formed as a leadless device, which does not comprise leads extending from a location outside of the heart into the heart for providing for a stimulation and/or sensing within the heart. The housing of the leadless pacemaker device may be placed on tissue with a distal end formed by the housing, the anchoring device and the electrode device beneficially being placed on the distal end and engaging with tissue when placing the leadless pacemaker device on tissue with its distal end.

In one embodiment, the anchoring device is formed by a helically extending coil element. The anchoring device hence has the shape of a screw which may be screwed into tissue in order to establish an anchoring of the body of the implantable medical device to tissue. The anchoring device herein may be fixedly arranged on the body, such that the anchoring device extends from the body and may be brought into engagement with tissue by, for example, rotating the body as a whole and hence screwing the anchoring device into tissue. The anchoring device, in one embodiment, is electrically passive and hence does not carry electrodes. In another embodiment it however is also conceivable that one or multiple electrodes are placed on the anchoring device to provide for an additional coupling to tissue.

In one embodiment, the electrode device is arranged radially within the anchoring device. Herein, in one embodiment the electrode device may be arranged concentrically within the anchoring device and may be moved within the anchoring device in order to adjust the relative position of the electrode device with respect to the body. The anchoring device may have a wider diameter than the electrode device, such that the electrode device extends through the anchoring device and, by screwing the electrode device into tissue, may be axially moved with respect to the anchoring device.

In one embodiment, the anchoring device in the shape of the helically extending coil comprises a first sense of rotation in which the anchoring device is to be screwed into tissue, for example by moving the body as a whole. Herein, in one embodiment, the helically extending coil body of the electrode device comprises a second sense of rotation, which may be opposite to the first sense of rotation, such that the electrode device may be screwed into tissue in a rotational direction opposite to the sense of rotation of the anchoring device. In this way it can be prevented that a screwing of the helically extending coil body of the electrode device into tissue by moving the electrode device with respect to the body causes a dislocation of the anchoring device, potentially leading to a loosening of the body from the tissue. In that the anchoring device and the electrode device comprise an opposite sense of rotation, rotating the electrode device to screw the electrode device into tissue causes a load on the anchoring device in the direction of the first sense of rotation, such that the anchoring device is held in tight engagement with tissue when rotating the electrode device for screwing the helically extending coil body of the electrode device into tissue.

In another embodiment, the anchoring device is formed by a flexibly bendable tine. A tine of this kind may in particular be arranged on the body with a first end and may extend from the body in a distal direction to form an apex and to bend backwards generally opposite to the distal direction to form a hook. When being placed on tissue, the tine may engage with tissue such that the apex is placed within tissue, a second, far end of the tine, for example, being placed outside of the tissue such that the tine forms a hook to anchor the implantable medical device to tissue at a location of interest. By flexibly bending the tine each anchoring device may be brought into engagement with tissue, wherein upon deployment each tine flexibly deforms and resets towards its original, unbent shape in order to form a hook for anchoring the implantable medical device to the tissue.

In yet another embodiment, the anchoring device may be formed from one or multiple spike elements, from one or multiple hooks or from one or multiple other engagement elements arranged on the body in order to provide for an anchoring engagement with tissue.

In one embodiment, the at least one electrode of the electrode device is placed at a tip of the helically extending coil body. The helically extending coil body may, for example, be formed from a metal core which is covered with an electrically insulating coating. The area of the electrode herein is left free such that the electrically conductive core of the helically extending coil body may come into electric contact with surrounding tissue at the location of the electrode.

In another embodiment, the electrode device may be formed from an electrically non-conductive helically extending coil body, on which at least one electrode element is placed, in particular in the region of the tip of the helically extending coil body. An electrical conductor herein may be embedded in the helically extending coil body to contact the electrode placed at the outside of the helically extending coil body.

In one embodiment, the electrode device comprises a pin to which the helically extending coil body is connected and about which the helically extending coil body extends. The helically extending coil body hence is arranged on the pin and extends helically around the pin to form a threading on the pin. The helically extending coil body may extend over an axial portion of the pin or along the entire length of the pin. In one embodiment, the helically extending coil body protrudes axially beyond a tip of the pin. One or multiple electrodes herein may be placed on the pin. Alternatively or in addition, one or multiple electrodes may be placed on the helically extending coil body.

In one embodiment, the electrode device is connected to an inner conductor received within the body, wherein the electrode device is movable with respect to the body together with the inner conductor. The inner conductor may be embedded in a tubing in order to provide for an electrical insulation. The inner conductor is received within the body and is movable, in particular rotatable, with respect to the body in order to move the electrode device with respect to the body.

The inner conductor may, for example, have the shape of a wire, a cable or a helically wound coil. The inner conductor may, for example, be fabricated from a cable, for example, made from a nickel cobalt alloy, such as MP35N.

In one embodiment, the inner conductor forms the electrode device. In particular, a pin on which one or multiple electrodes are placed may be formed by the inner conductor. A distal end of the inner conductor hence may form the electrode device, wherein the helically extending coil body may be fixed to the distal end of the inner conductor, for example by gluing or welding, or may be integrally formed with the distal end of the inner conductor, for example by stamping, rolling or milling. In that the electrode device is immediately formed on the inner conductor, an easy, robust design of the electrode device is obtained.

The inner conductor may be electrically insulated in the region of the electrode device, wherein the coating is interrupted at one or multiple locations in order to form one or multiple electrodes on the electrode device at the distal end of the inner conductor.

In one embodiment, an insulation is formed on the inner conductor proximally with respect to the helically extending coil body. Herein, the insulation may have a diameter which is equal to or larger than the diameter of the helically extending coil body. This may allow to form a tissue channel in the tissue when inserting the inner conductor with its insulation into tissue, the tissue channel being formed proximally of the helically extending coil body and assuming the diameter of the insulation. This may ease the extraction of the electrode device during explantation (if necessary), because the helically extending coil body may easily be moved within the channel formed by the large diameter insulation.

The inner conductor may be connected to a connector at the proximal end of a lead (in case the body is formed by a lead to be connected to a generator) in that the inner conductor is electrically contacted to a contact element of the connector.

If the electrode device is connected to or formed by an inner conductor, the electrode device may be moved by rotating the inner conductor. A rotational movement of the electrode device with its helically extending coil body causes the electrode device to be screwed into tissue, pulling the inner conductor behind such that an electrically coupling to tissue is obtained.

In another embodiment, in particular when the electrode device is not movable by means of an inner conductor, a mandrel or another tool to be inserted into the body may be used to move the electrode device.

In one embodiment, the electrode device is movable with respect to the body through a channel formed in a housing element. The body may generally extend along a longitudinal axis. The channel herein may extend straight, or may have a curved shape. For example, the channel may form a 90° curve. In particular in embodiments in which the electrode device is made from an elastically bendable material, such as from a wire or the like, a bent shape of the channel may cause the electrode device to exit from the body along a direction which differs from the longitudinal axis along which the body extends and along which, for example, an inner conductor connected to the electrode device is movable inside the body. In the engagement position, hence, the electrode device may extend and protrude from the body along a direction which is different, for example perpendicular, to the longitudinal axis along which the body extends.

In one embodiment, the body comprises a housing element in which the electrode device is received. Herein, one of the housing element and the electrode device comprises a threading and the other of the housing element and the electrode device comprises a counter element engaging with the threading such that a rotational movement of the electrode device relative to the housing element about a longitudinal axis causes a linear displacement of the electrode device relative to the housing element along the longitudinal axis. The electrode device hence is coupled to the body by means of a screwing mechanism comprising a threading and a counter element. By rotating the threading with respect to the counter element due to a rotational movement of the electrode device with respect to the body the electrode device may be axially moved with respect to the body such that the electrode device may be brought into engagement with tissue or, for explantation, may be moved out of engagement with tissue.

In one embodiment, the implantable medical device comprises a processing device configured to control operation of the at least one electrode for at least one of emitting an electrical stimulation signal and sensing an electrical sense signal using the at least one electrode. The processing device may be part of a generator, in case the body is formed by a lead extending from the generator. Alternatively, the processing device may be part of a leadless pacemaker device forming the implantable medical device. The processing device may control operation of the implantable medical device, in that, for example, one or multiple electrodes are selected in order to capture the left bundle branch and to provide for a stimulation or sensing at the left bundle branch using one or multiple electrodes.

In one embodiment, the implantable medical device comprises one or multiple further electrodes, such as an electrode arranged on the body, for example in the shape of a ring electrode or a coil electrode, for example serving as a counter electrode for the at least one electrode arranged on the electrode device, or as a shock electrode for providing a defibrillation function.

Alternatively or in addition, one or multiple sensors, such as temperature sensors or pressure sensors or the like may be arranged on or integrated in the body.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

An idea of the present invention shall subsequently be described in more detail with reference to the embodiments shown in the figures. Herein:

FIG. 1 shows a schematic view of the human heart, including the Sinoatrial node, the Atrioventricular node, the HIS bundle and the left bundle branch and right bundle branch extending from the HIS bundle;

FIG. 2 shows a schematic drawing of the heart with a lead implanted therein;

FIG. 3 shows a schematic drawing of the heart with a leadless stimulation device implanted therein;

FIG. 4 shows a view of an embodiment of an implantable medical device having a body and an anchoring device as well as an electrode device arranged thereon;

FIG. 5 shows an enlarged view of a distal end of the implantable medical device with the anchoring device and the electrode device;

FIG. 6A shows the implantable medical device, with the electrode device in a retracted position;

FIG. 6B shows the implantable medical device, with the electrode device in an engagement position;

FIG. 7 shows a view of another embodiment of an implantable medical device;

FIG. 8 shows a view of an embodiment of an implantable medical device with an electrode device formed by an inner conductor;

FIG. 9 shows a view of another embodiment of an implantable medical device;

FIG. 10 shows another embodiment of an electrode device;

FIG. 11A shows the implantable medical device of FIG. 8, in a retracted position of the electrode device;

FIG. 11B shows the implantable medical device of FIG. 8, in an engagement position of the electrode device;

FIG. 12 shows a view of an embodiment of a connector for connecting a lead to a generator;

FIG. 13 shows another embodiment of a connector;

FIG. 14 shows a view of an embodiment of an implantable medical device, with an electrode device movable along a perpendicular direction with respect to a longitudinal direction of extension of a lead of the implantable medical device;

FIG. 15 shows a schematic, sectional view of the implantable medical device of FIG. 14;

FIG. 16A shows another embodiment of an implantable medical device, with an electrode device in a retracted position; and

FIG. 16B shows the embodiment of FIG. 16A, in an engagement position of the electrode device.

### DETAILED DESCRIPTION

Subsequently, embodiments of the present invention shall be described in detail with reference to the drawings. In the drawings, like reference numerals designate like structural elements.

It is to be noted that the embodiments are not limiting for the present invention, but merely represent illustrative examples.

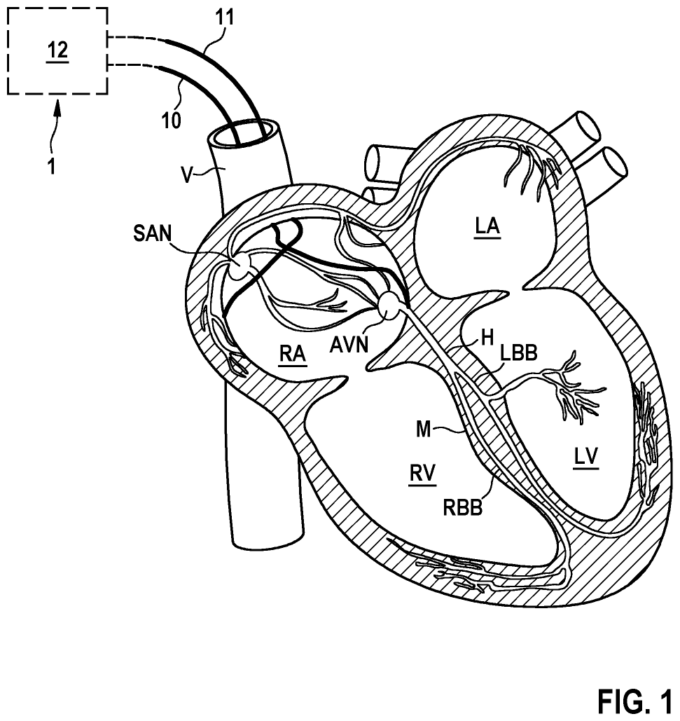

FIG. 1 shows, in a schematic drawing, the human heart comprising the right atrium RA, the right ventricle RV, the left atrium LA and the left ventricle LV, the so-called sinoatrial node SAN being located in the wall of the right atrium RA, the sinoatrial node SAN being formed by a group of cells having the ability to spontaneously produce an electrical impulse that travels through the heart's electrical conduction system, thus causing the heart to contract in order to pump blood through the heart. The atrioventricular node AVN serves to coordinate electrical conduction in between the atria and the ventricles and is located at the lower back section of the intra-atrial septum near the opening of the coronary sinus. From the atrioventricular node AVN the so-called HIS bundle H is extending, the HIS bundle H being comprised of heart muscle cells specialized for electrical conduction and forming part of the electrical conduction system for transmitting electrical impulses from the atrioventricular node AVN via the so-called right bundle branch RBB around the right ventricle RV and via the left bundle branch LBB around the left ventricle LV.

In the embodiment of FIG. 1, an implantable medical device 1 in the shape of a stimulation device, such as a CRT device, is implanted in a patient, the implantable medical device 1 comprising a generator 12 connected to leads 10, 11 extending from the generator 12 through the superior vena V into the patient's heart. By means of the leads 10, 11, electrical signals for providing a pacing action in the heart shall be injected into intra-cardiac tissue potentially at different locations within the heart, and sense signals may be received. In addition, possibly a defibrillation therapy may be performed by an electrode arrangement arranged on one or both of the leads 10, 11.

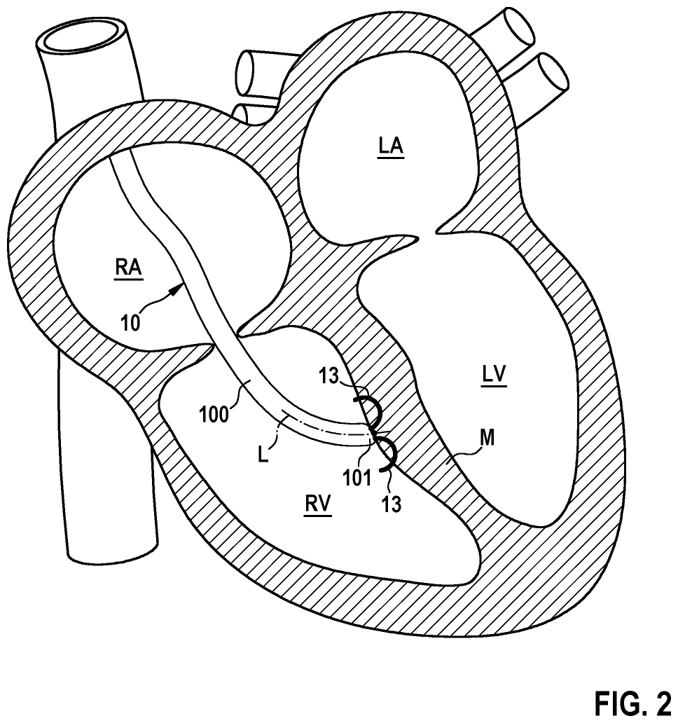

In an embodiment shown in FIG. 2, a lead 10 is implanted into the heart such that it extends into the right ventricle RV of the heart and, at a distal end 101 of a lead body 100, is arranged on intra-cardiac tissue at the septum M in between the right ventricle RV and the left ventricle LV of the heart. At the distal end 101 herein anchoring devices 13 in the shape, e.g., of tines are arranged, the anchoring devices 13 serving to anchor the lead 10 with its body 100 to tissue in particular in the region of the septum M within the heart.

An implantable medical device 1 as concerned herein may generally be a cardiac stimulation device such as a cardiac pacemaker device. A stimulation device of this kind may comprise a generator 12, as shown in FIG. 1, which may be subcutaneously implanted into a patient at a location remote from the heart, one or multiple leads 10, 11 extending from the generator 12 into the heart for emitting stimulation signals in the heart or for obtaining sense signals at one or multiple locations from the heart.

If the implantable medical device 1 is a stimulation device using leads, a lead 10 forms a generally longitudinal, tubular body 100 extending along a longitudinal axis L, as shown in FIG. 2, which reaches into the heart and is anchored at a location of interest, for example, on the septum M of the heart in the region of the right ventricle RV.

In another embodiment, the implantable medical device 1 may be a leadless pacemaker device, which does not comprise leads, but has the shape of a capsule and may be directly implanted into the heart, for example into the right ventricle RV of the heart.

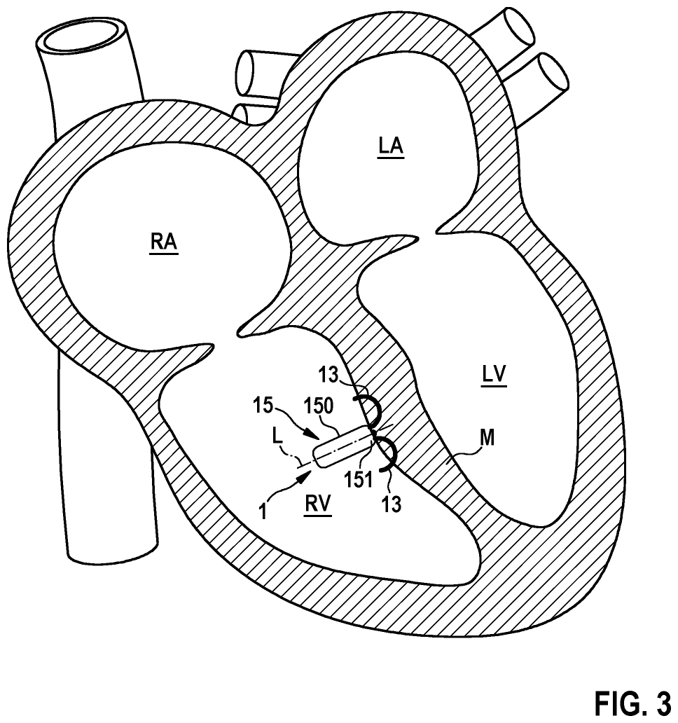

Referring now to FIG. 3, in one embodiment an implantable medical device 1 in the shape of a leadless pacemaker device 15 comprises a body 150 in the shape of a housing which extends longitudinally along a longitudinal axis L and encapsulates components of the leadless pacemaker device 15, such as a processing device, a data memory, a battery, pulse generation circuitry and the like to allow for a stimulation operation immediately within the heart.

In the embodiment of FIG. 3, the body 150 in the shape of the housing of the leadless pacemaker device 15 forms a distal end 151 which is placed on intra-cardiac tissue in the region of the septum M of the heart. Anchoring devices 13 in the shape, e.g., of tines are arranged on the body 150 in the region of the distal end 151 and extend from the distal end 151 generally along the longitudinal axis L.

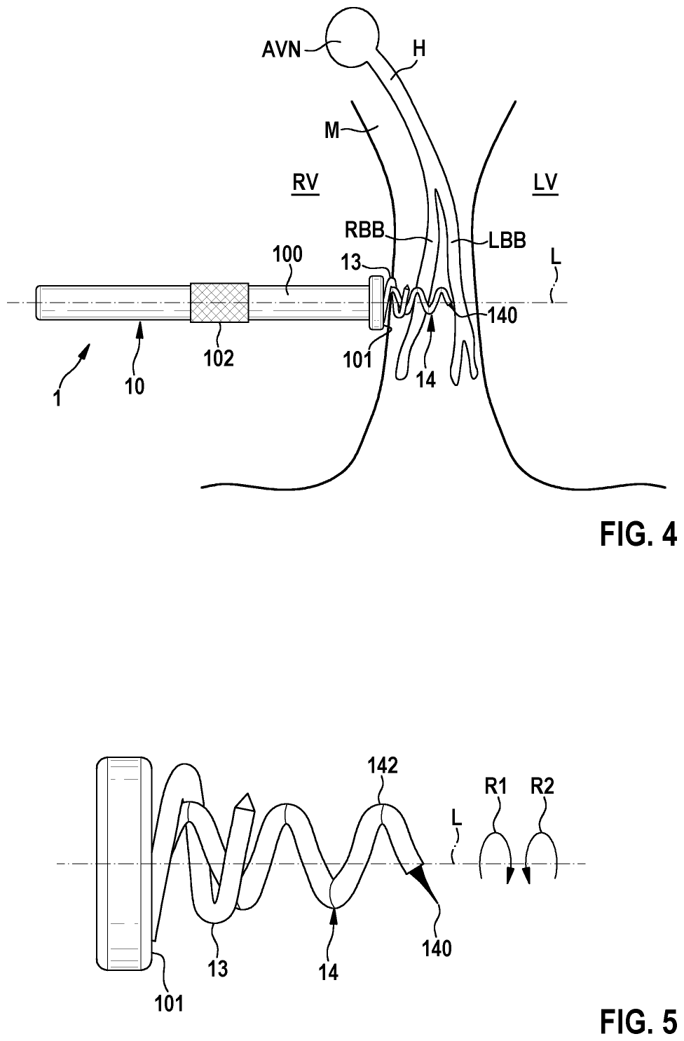

Referring now to FIGS. 4 to 6A, and 6B, in one embodiment an implantable medical device 1 comprises a lead 10 having a body 100 which, at a distal end 101, is to be placed on tissue, for example, on the septum M of the heart.

In the shown embodiment, an anchoring device 13 in the shape of a helically wound coil is arranged on the distal end 101, the anchoring device 13 being configured to anchor the body 100 at its distal end 101 to the tissue in the region of the septum M. The anchoring device 13 herein is fixedly arranged on the distal end 101 and serves the function of a screw which may be screwed into tissue by rotating the body 100 as a whole in a sense of rotation R1, as this is indicated in FIG. 5.

In addition, an electrode device 14 having a helically extending coil body 142 is arranged on the body 100 to extend from the body 100 at the distal end 101. The electrode device 14 is axially movable along a longitudinal axis L, along which the body 100 generally extends, with respect to the body 100.

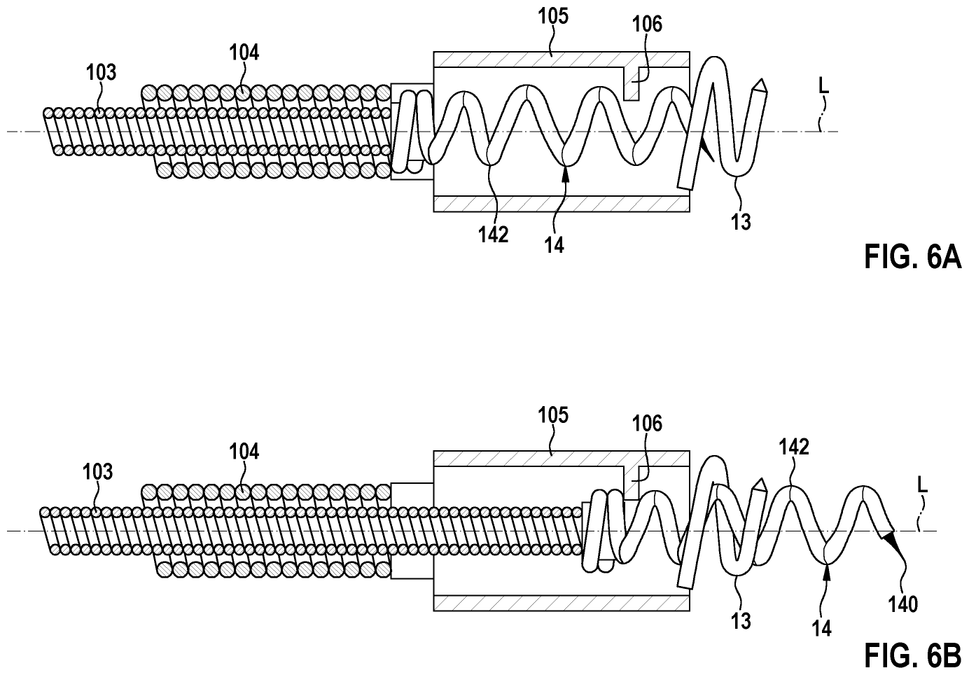

As this is shown in FIGS. 6A and 6B, the electrode device 14 with its helically extending coil body 142 is connected to an inner conductor 103 received within an outer conductor 104 of the body 100. The outer conductor 104 is fixedly connected to a housing element 105, which may, for example, form the distal end 101 of the body 100. The inner conductor 103 is movable, i.e. rotatable and axially slidable, within the outer conductor 104.

Both the inner conductor 103 and the outer conductor 104 may be embedded in an electrically insulating tubing material, which in FIGS. 6A and 6B is not shown for the sake of easy illustration.

The electrode device 14 with its helically extending coil body 142 is rotatable by rotating the inner conductor 103. Inside of the housing element 105 herein a counter element 106, e.g., in the shape of a stud protruding radially inwards is formed, the counter element 106 engaging with the helically extending coil body 142 such that a rotation of the helically extending coil body 142 causes an interaction with the counter element 106 and hence an axial displacement of the electrode device 14 and the inner conductor 103 along the longitudinal axis L with respect to the housing element 105 and the body 100 connected thereto.

The electrode device 14 comprises an electrode 140 formed by a pointed tip of the helically extending coil body 142. The helically extending coil body 142 may, for example, be formed from an electrically conductive core which is coated by an electrically insulating coating material, wherein the helically extending coil body 142, for example, does not comprise a coating in the region of the tip, such that the electrode 140 is formed at the tip by exposing the electrically conductive inner core of the helically extending coil body 142.

With its electrode 140, hence, the electrode device 14 may come into engagement with tissue in order to electrically couple to tissue for emitting electrical stimulation signals into and/or receiving electrical sense signals from tissue.

The electrode device 14, as apparent from FIGS. 6A and 6B, may be moved in between different positions with respect to the housing element 105 of the body 100. In a retracted position, shown in FIG. 6A, the electrode device 14 fully or at least with substantial portions is received within the housing element 105, such that the electrode device 14 does not or only with a portion extends from the housing element 105. By rotating the inner conductor 103 the electrode device 14 may be moved out of the housing element 105, as this is shown in FIG. 6B, in order to bring the electrode device 14 into engagement with tissue at a location of interest.

Herein, the electrode device 14 may be continuously moved such that the electrode device 14 may be brought into different positions to engage with a particular tissue region at a particular depth. This may be used, as indicated in FIG. 4, in particular to provide for a pacing at the left bundle branch LBB. In particular, by screwing the electrode device 14 into tissue the electrode 140 may be brought into a position in which it is located in the vicinity of the left bundle branch LBB, such that the electrode 140 of the electrode device 14 may couple with the left bundle branch LBB and hence may capture the left bundle branch LBB to inject signals into the left bundle branch LBB and/or receive sense signals from the left bundle branch LBB.

As the electrode device 14 may assume different positions with respect to the body 100 and hence may be variably moved to a particular depth, the electrode 140 may be placed at different depths within tissue and hence may provide for an excitation and/or sensing at a particular location depending on the position of the electrode device 14. In particular, during implantation a capturing of the electrode 140 to tissue, in particular to the left bundle branch LBB, may be monitored, such that the electrode device 14 may be brought into a position in which an optimum coupling to the left bundle branch LBB may be established, such that an effective stimulation and/or sensing at the left bundle branch LBB may be achieved.

As it is illustrated in FIG. 5, in one embodiment the electrode device 14 with its helically extending coil body 142 comprises a sense of rotation R2 which is opposite to the sense of rotation R1 of the anchoring device 13. This allows to screw the anchoring device 13 into tissue in the sense of rotation R1, while the electrode device 14 is in its retracted position according to FIG. 6A. Once the body 100 with its distal end 101 is coupled to tissue by means of the anchoring device 13, the electrode device 14 may be screwed into tissue in the sense of rotation R2, which causes a load on the anchoring device 13 in the sense of rotation R1 such that the anchoring by means of the anchoring device 13 is tightened rather than loosened when screwing the electrode device 14 into tissue for bringing the electrode 140 into a position in which an effective coupling to tissue is established.

In the embodiment of FIGS. 4 to 6A, and 6B, the electrode device 14 is formed by a helically extending element which is fixedly connected to the inner conductor 103, which itself is formed by a helically extending, coil-shaped electrical conductor.

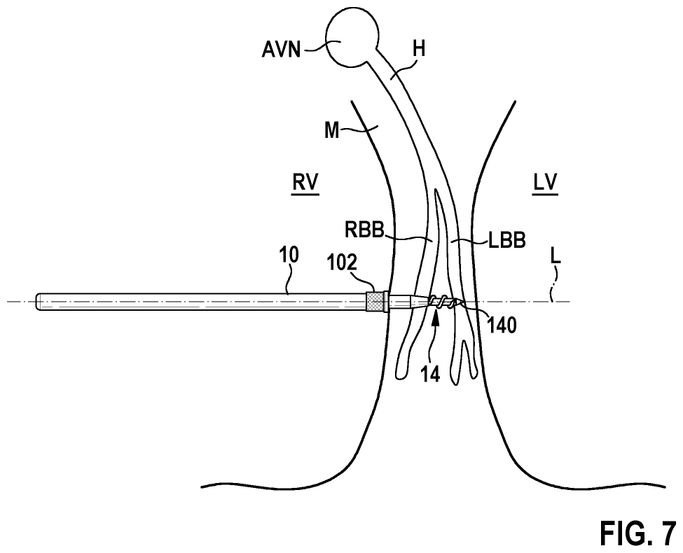

In the embodiments of FIGS. 7 to 11A, and 11B, the electrode device 14 is formed by the inner conductor 103 itself. The electrode device 14 herein comprises a pin 141 formed by an inner core of the inner conductor 103, which may, for example, be formed by a wire or a rope, for example from a nickel cobalt alloy, such as MP35N. A helically extending coil body 142 is arranged on the pin 141. The helically extending coil body 142 may, for example, be formed by a separate element which is glued or welded to the pin 141. Alternatively, the helically extending coil body 142 may be integrally formed with the pin 141, for example by a stamping, rolling or milling technique.

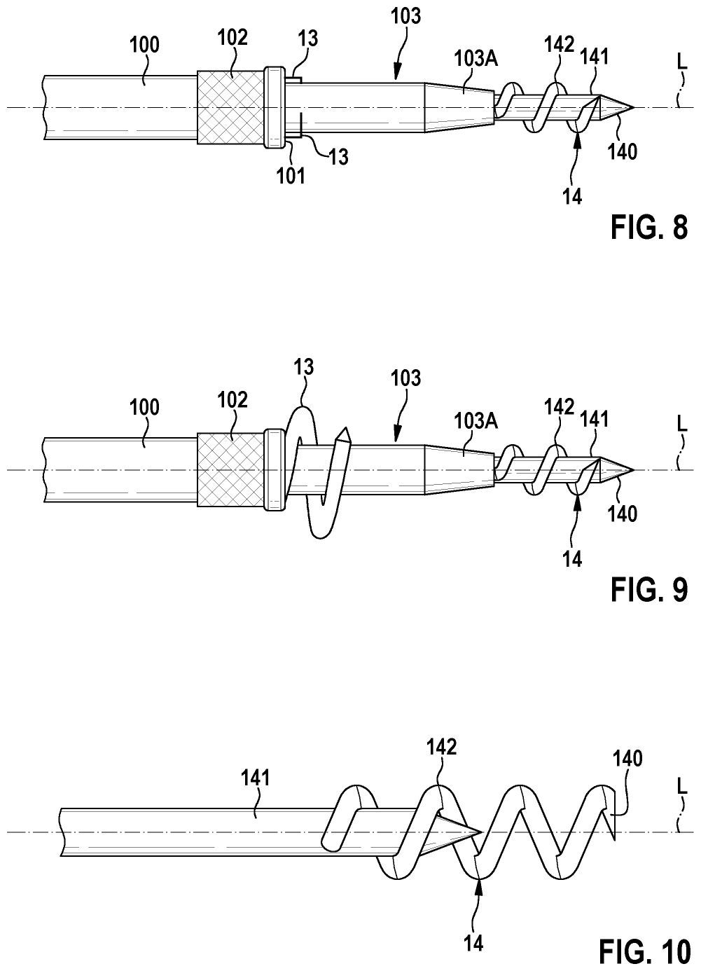

FIGS. 8 to 10 show different modifications of the implantable medical device 1.

In the embodiment of FIG. 8, the inner conductor 103 comprises an inner core, for example made from a conductive wire, which forms the pin 141. An electrical insulation 103A is placed on the inner core and surrounds the inner core, wherein the pin 141 is not covered by the electrical insulation 103A. In the region of the pin 141 an electrically insulating, thin coating may be placed, wherein a tip of the pin 141 is left free such that an electrode 140 is formed at the tip of the pin 141 and exposed towards the outside.

A helically extending coil body 142 extends about the pin 141, wherein the helically extending coil body 142 may be integrally formed with the pin 141, or may be glued or welded to the pin 141 as a separate element.

As visible from FIG. 8, the helically extending coil body 142 comprises a diameter (measured transverse to the longitudinal axis L) which is equal to or smaller than the diameter of the electrical insulation 103A of the inner conductor 103 proximally of the helically extending coil body 142. In this way the electrode device 14 may be screwed into tissue, wherein the electrical insulation 103A forms a channel within the tissue having the diameter of the electrical insulation 103A. This may facilitate an explantation (if necessary) in that for explantation the helically extending coil body 142 may be unscrewed, and once the tissue channel formed by the electrical insulation 103A is reached the helically extending coil body 142 may be slid out of the channel by pulling on the body 100.

In the embodiment of FIG. 8, the anchoring device 13 is formed by hook elements placed on the distal end 101 of the body 100.

A further electrode 102 in the shape of a ring electrode may be placed on the body 100.

In the embodiment of FIG. 9, the electrode device 14 and the inner conductor 103 are identical in shape and function to the electrode device 14 and the inner conductor 103 of the embodiment of FIG. 8. In contrast to the embodiment of FIG. 8, the anchoring device 13 in the embodiment of FIG. 9 is formed by a helically extending screw element, which is fixedly arranged on the body.

The helically extending screw element 13 may have a sense of rotation which may be equal to or opposite to a sense of rotation of the helically extending coil body 142 of the electrode device 14.

Whereas in the embodiments of FIGS. 8 and 9 the helically extending coil body 142 is arranged on the pin 141 formed by the inner conductor 103 to extend substantially along the length of the pin 141, in the embodiment of FIG. 10 the helically extending coil body 142 protrudes beyond the tip of the pin 141 along the longitudinal axis L. An electrode 140 herein is formed by the helically extending coil body 142. The pin 141 may entirely be covered by an electrically insulating coating, such that the pin 141, for example, does not electrically couple to tissue.

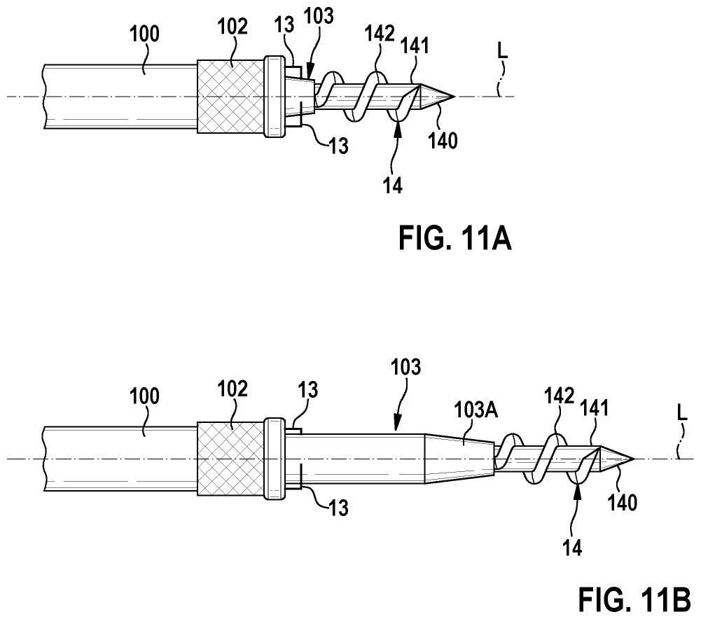

As visible from FIGS. 11A and 11B, the electrode device 14 may be placed in tissue at different depths, such that the electrode 140 may be brought into a position at a particular depth in which an efficient coupling to a particular tissue region may be established.

In the embodiments of FIGS. 7 to 11A, and 11B, the inner core of the conductor 103 may be formed by wire filaments, for example, made from a nickel cobalt alloy. The wire filaments form the pin 141 and may be welded together to form the distal tip of the pin 141. In regions of the pin 141 other than the electrode region 140 an electrically insulating coating, such as a parylene coating, may be placed to electrically insulate the pin 141 in regions not covered by the electrical insulation 103A.

By forming the electrode device 14 integrally with the inner conductor 103, an easy, robust arrangement can be provided. In particular, a risk of breaking at a transition from the electrode device 14 to the inner conductor 103 is substantially reduced, in that the electrode device 14 is integrally formed with the inner conductor 103. The inner conductor 103 is movable together with the electrode device 14, such that the inner conductor 103 may be moved into tissue together with the electrode device 14, allowing to place the electrode device 14 at different depths within tissue.

A further electrode 102 placed on the body 100 may form a counter electrode for the electrode 140 of the electrode device 14. In addition or alternatively, the further electrode 102 may form a shock electrode, for example for providing a defibrillation function. The further electrode 102 may have the shape e.g. of a ring electrode or a coil electrode.

The implantable medical device 1 may be formed, e.g., by a stimulation device having a lead, as shown in FIGS. 1 and 2. Alternatively, the implantable medical device 1 may have the shape of a leadless pacemaker, as shown in FIG. 3.

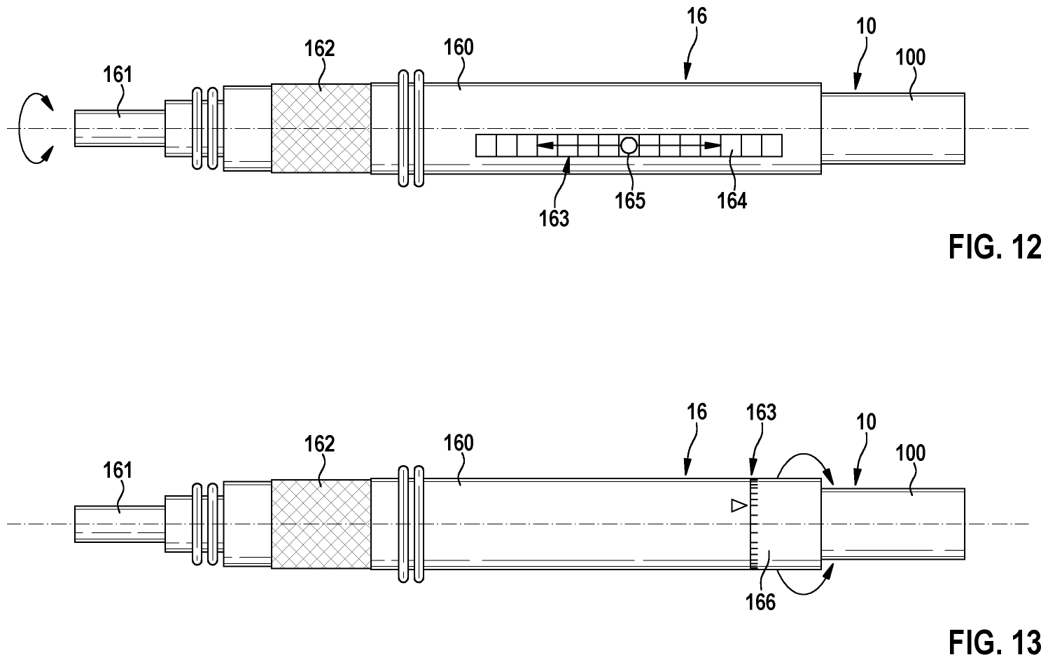

If the implantable medical device 1 is formed by a device having a lead 10, the lead 10 at its proximal end may have a connector 16 as shown in FIG. 12, the connector 16 having contact elements 161, 162 arranged on a connector body 160 for electrically connecting the lead 10 to a generator 12 by plugging the connector 16 into a corresponding plug receptacle of the generator 12. The connector 16 may have a standardized shape and may form a DF4 or DF2 connector.

Herein, as indicated in FIG. 12, the connector 16 may comprise a gauge device 163 having a window 164 in which a marker element 165 is movable to indicate a relative position of the electrode device 14 with respect to the body 100 of the lead 10. The marker element 165 may be mechanically coupled to the inner conductor 103 received within the body 100, such that a relative position of the marker element 165 in the window 164 indicates the position of the electrode device 14 with respect to the body 100 and in particular a depth of an electrode 140 provided by the electrode device 14.

In the embodiment of FIG. 12, the inner conductor 103 may, for example, be rotated by rotating the contact element 161, as illustrated in FIG. 12. The contact element 161 herein may be elastically coupled to the inner conductor 103, such that a rotation of the contact element 161 causes a rotation of the inner conductor 103, wherein an axial displacement of the inner conductor 103 with respect to the contact element 161 is compensated by the elasticity of the connection of the inner conductor 103 to the contact element 161.

In another embodiment of a connector 16 shown in FIG. 13, the connector 16 comprises a turning wheel 166 which is connected to the inner conductor 103 received in the body 100, such that by rotation of the turning wheel 166 the inner conductor 103 may be rotated and the electrode device 14 connected to the inner conductor 103 may be moved within the body 100. A scale herein may be provided at a transition in between the turning wheel 166 and the connector body 160 of the connector 16, such that the relative position of the turning wheel 166 indicates the position of the electrode device 14 with respect to the body 100.

In the embodiment of FIG. 13, the body 100 with its outer conductor 104 may be elastically coupled to the connector body 160 and the contact element 162, to which the outer conductor 104 is electrically contacted. If the turning wheel 166 is rotated, the body 100 may be axially moved with respect to the connector body 160, wherein the body 100, for example, is supported on the connector body 160 by means of a spring element, which, for example, also provides for an electrical connection of the outer conductor 104 of the body 100 with the contact element 162.

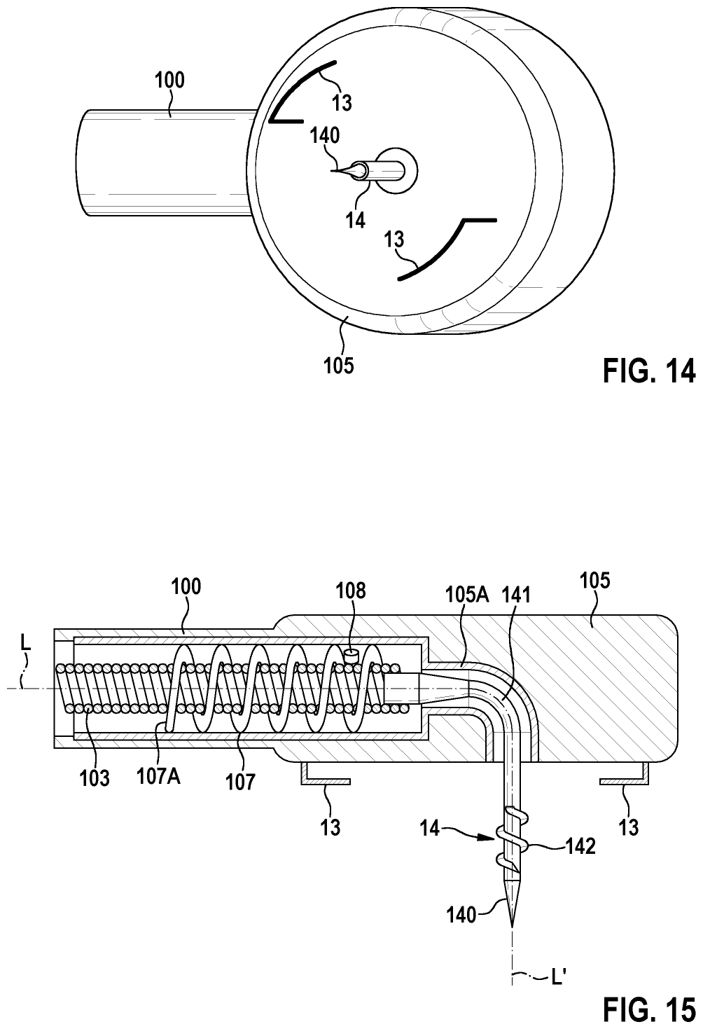

In an embodiment shown in FIGS. 14 and 15, the body 100 forms a housing element 105 which comprises a channel 105A having a curved shape through which the electrode device 14 extends, as visible from FIG. 15. The electrode device 14, formed, for example, from an elastically deformable wire or formed integrally with the inner conductor 103, may be elastically bent such that, by moving the inner conductor 103, the electrode device 14 may be moved out of the housing element 105.

In the embodiment of FIGS. 14 and 15, the channel 105A forms a 90° angle, such that an axis L′ along which the electrode device 14 is moved out of the housing element 105 extends perpendicularly to the longitudinal axis L along which the body 100 generally extends. The engagement direction of the electrode device 14 hence differs from the longitudinal direction of extension of the body 100.

The electrode device 14 may comprise a helically extending coil body 142 and forms a tip electrode 140, allowing to screw the electrode device 14 into tissue in order to electrically couple the electrode 140 to tissue.

In the embodiment of FIGS. 14 and 15, the body 100, in the region of the housing element 105 forming the distal end of the body 100, comprises a drive cylinder 107 in which a threading 107A is formed. The inner conductor 103 herein comprises a counter element 108 engaging with the threading 107A, such that a rotation of the inner conductor 103 causes an axial displacement of the electrode device 14 with respect to the housing element 105.

In the embodiment of FIGS. 14 and 15 the helically extending coil body 142 may also be dispensable, the electrode device 14 hence being formed solely by the pin 141 which may be inserted into tissue by advancing the electrode device 14 by rotating the inner conductor 103.

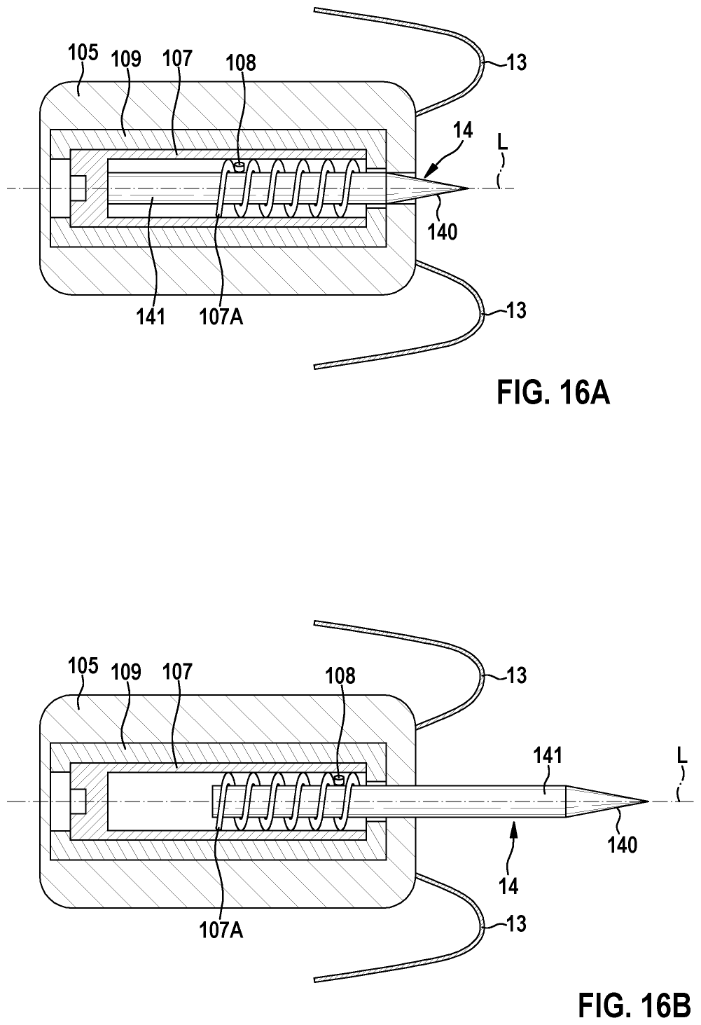

In an embodiment shown in FIGS. 16A and 16B an electrode device 14 is formed by a pin 141 forming an electrode 140. The electrode device 14 is operatively coupled to a drive cylinder 107, which is rotatable within a bearing element 109 fixedly arranged within the housing element 105. The pin 141 of the electrode device 14 comprises a counter element 108 in the shape of a stud, which engages with a threading 107A formed inside the drive cylinder 107. A rotation of the drive cylinder 107 within the bearing element 109 hence causes the electrode device 14 to be axially advanced with respect to the housing element 105, as it is visible from the different positions of the electrode device 14 with respect to the housing element 15 in FIGS. 16A and 16 B.

In the embodiment of FIGS. 16A, 16B, the anchoring device 13 is formed by tines arranged on the housing element 105.

A rotation of the drive cylinder 107 may, for example, be achieved by a mandrel, which may be brought into operative connection with the drive cylinder 107, for example through a body 100 of a lead 10 or, in case of a leadless pacemaker, by engaging the mandrel with the leadless pacemaker during implantation.

The idea of the present invention is not limited to the embodiments described above.

The implantable medical device may have the shape of a stimulation device comprising leads, or may have the shape of a leadless pacemaker device.

The electrode device may be formed in different ways and is not limited to the embodiments described above. The electrode device may be integrally formed with an inner conductor. The electrode device, alternatively, may be formed by an element connected to an inner conductor.

The electrode device may be rotated by an inner conductor. Alternatively, the electrode device may be rotated by a mandrel or the like which may be operatively connected to the electrode device.

The electrode device may comprise one or multiple electrodes. If multiple electrodes are placed on the electrode device, the electrodes may be electrically independent of each other, such that one or multiple electrodes of the electrode device may be used separately or in combination to provide for an electrical stimulation and/or electrical sensing at a location of interest.

The anchoring device may be formed in different ways and is not limited to the embodiments described above. Generally, the anchoring device may be formed by hooks, tines or one or multiple screw elements. Elements of the anchoring device may be rigid, or may be elastically deformable.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device

10 Lead

100 Lead body

101 Distal end

102 Electrode

103 Inner conductor

103A Electrical insulation

104 Outer conductor

105 Housing element

105A Channel

106 Counter element

107 Drive cylinder

107A Threading

108 Counter element

109 Bearing element

11 Lead

12 Generator

13 Anchoring device

14 Electrode device

140 Electrode

141 Pin

142 Coil body

15 Leadless device

150 Body (housing)

151 Distal end

16 Connector

160 Connector body

161, 162 Contact element

163 Gauge device

164 Window

165 Marker element

166 Turning wheel

AVN Atrioventricular node

H HIS bundle

L, L′ Longitudinal axis

LA Left atrium

LBB Left bundle branch

LV Left ventricle

M Intra-cardiac tissue (myocardium)

R1, R2 Sense of rotation

RA Right atrium

RBB Right bundle branch

RV Right ventricle

SAN Sinoatrial node

V Superior vena

1. An implantable medical device for implantation into a patient, comprising:
a body,
an anchoring device for anchoring the body to tissue at a location of interest, the anchoring device being arranged on the body, and
an electrode device for at least one of emitting an electrical stimulation signal and sensing an electrical sense signal,
wherein the electrode device comprises at least one electrode and a helically extending coil body wherein the electrode device is movable with respect to the body between a retracted position, in which the electrode device at least partially is received within the body, and an engagement position, in which the electrode device is moved to protrude from the body to engage with tissue.

2. The implantable medical device according to claim 1, wherein the body forms a distal end to be placed on tissue upon implantation of the implantable medical device, wherein the anchoring device is arranged on and extends from the distal end and wherein the electrode device is movable with respect to the distal end.

3. The implantable medical device according to claim 1, wherein the electrode device is rotatable with respect to the body to screw said helically extending coil body into tissue.

4. The implantable medical device according to claim 1, wherein the body is formed by a lead which is connectable to a generator of the implantable medical device.

5. The implantable medical device according to claim 4, wherein the lead comprises a connector which the lead is connectable to said generator, wherein the connector comprises a gauge device for indicating the position of the electrode device with respect to the body.

6. The implantable medical device according to claim 1, wherein the body is formed by a housing of a leadless pacemaker device.

7. The implantable medical device according to claim 1, wherein the anchoring device is formed by a helically extending coil.

8. The implantable medical device according to claim 7, wherein the electrode device is arranged concentrically within the anchoring device.

9. The implantable medical device according to claim 7, wherein the anchoring device formed by the helically extending coil comprises a first sense of rotation, and the helically extending coil body of the electrode device comprises a second sense of rotation opposite to the first sense of rotation.

10. The implantable medical device according to claim 1, wherein the anchoring device is formed by at least one flexibly bendable tine.

11. The implantable medical device according to claim 1, wherein the at least one electrode is placed at a tip of the helically extending coil body.

12. The implantable medical device according to claim 1, wherein the electrode device comprises a pin to which the helically extending coil body connected and about which the helically extending coil body extends.

13. The implantable medical device according to claim 1, wherein the electrode device is connected to an inner conductor received within the body, wherein the electrode device is movable with respect to the body together with the inner conductor.

14. The implantable medical device according to claim 13, wherein the inner conductor forms said at least one electrode.

15. The implantable medical device according to claim 1, wherein the body comprises a housing element in which the electrode device is received, wherein one of the housing element and the electrode device comprises a threading and the other of the housing element and the electrode device comprises a counter element engaging with the threading such that a rotational movement of the electrode device relative to the housing element about a longitudinal axis causes a linear displacement of the electrode device relative to the housing element along the longitudinal axis.
